(19) United States
(12) Patent Application Publication
(10) Pub. No.: US 2025/0001697 A1
(43) Pub. Date: Jan. 2, 2025

(54) ANATOMICALLY ACCURATE RODENT HEAD MODELS AND BRAIN PHANTOMS AND METHODS FOR MAKING AND USING THE SAME

(71) Applicant: VIRGINIA COMMONWEALTH UNIVERSITY, Richmond, VA (US)

(72) Inventors: Chirayu NIMONKAR, Glen Allen, VA (US); Eli KNIGHT, Williamsburg, VA (US); Ivan C. CARMONA, Richmond, VA (US); Ravi L. HADIMANI, Glen Allen, VA (US)

(21) Appl. No.: 18/575,488

(22) PCT Filed: Jul. 1, 2022
(86) PCT No.: PCT/US2022/036001

§ 371 Date: Dec. 29, 2023

Related U.S. Application Data
(60) Provisional application No. 63/217,972, filed on Jul. 2, 2021.

(51) Int. Cl. B29C 64/393; B29C 33/38; B29C 39/12; B33Y 50/02; B33Y 80/00; G06T 7/00; G06T 7/10

(52) U.S. Cl. CPC B29C 64/393; B29C 33/38; B29C 39/126; B33Y 50/02; B33Y 80/00; G06T 7/0014; G06T 7/10; G06T 2207/10081; G06T 2207/10088; G06T 2207/20112; G06T 2207/30016

(57) ABSTRACT

Anatomically accurate rodent brain models and phantoms, processes for constructing an anatomically correct computer simulation model of a rodent head, and fabricating the accurate rodent brain models and phantoms, and methods for leveraging the accurate rodent brain models and phantoms production are disclosed, and which may be used for simulated and experimental verification of induced electric fields and for experimentally testing neuromodulation and neuroimaging procedures.

### CROSS-REFERENCE TO RELATED APPLICATIONS

This application claims the benefit of U.S. Provisional Patent Application No. 63/217,972, filed Jul. 2, 2021, the complete contents of which are herein incorporated by reference.

### FIELD OF THE INVENTION

Embodiments generally relate to individualized, anatomically accurate rodent brain models and phantoms with separated tissues which can be used for simulated and experimental verification of induced electromagnetic fields in rodent brains, for the creation of new coils for transcranial magnetic stimulation (TMS) and other neuromodulation techniques, and for studies in rodents with neurological conditions such as Parkinson's disease.

### BACKGROUND

Transcranial Magnetic Stimulation (TMS) is a non-invasive technique that uses a time varying magnetic field to treat various neurological and psychiatric conditions. There are ethical and technical limitations in developing new TMS treatment procedures using human subjects, which can render TMS trials using human patients infeasible. Theoretically, without consideration of various time, cost, and anatomical accuracy shortcomings of current brain phantom design and production techniques, brain phantoms could be useable and practical as test vehicles for new TMS techniques. This would enable test and evaluation without potential risks from actual stimulating, patients' brain tissue with yet unproven TMS techniques. However, time, cost, and anatomical accuracy issues of current brain phantom production techniques are extant. For example, induced electric field or voltage in a rodent's brain during TMS has sensitivity to variations in brain anatomy, i.e., different tissues and head materials (such as grey matter, white matter, cerebrospinal fluid, skull, and scalp). However, in current techniques of modeling rodent brains, and in producing rodent brain phantoms, these different tissues and TMS sensitivities to such tissues' variations have not been considered.

A result of such issues includes a less-than-desired availability of useable brain phantoms. This in turn has caused, for example, a less than fully understood, there is shortcomings exist, is a lack of anatomically realistic head/brain phantoms. The lack of anatomically realistic brain phantoms has made the experimental verification of induced electromagnetic fields in the brain tissues an impediment to the development of new treatment protocols.

### SUMMARY

According to one or more embodiments, an example method can provide a constructing of an anatomically correct model of a rodent head that is useable for testing or evaluating a transcranial magnetic stimulation (TMS) or other neuromodulation system or method, and useable for fabricating a brain phantom that mimics the rodent head. The example method can include receiving, and storing in a data memory of a programmable computer resource, a tissue imaging data for the rodent head, the tissue imaging data including a magnetic resonance imaging (MRI) rodent head image data and a computer tomography (CT) imaging rodent head data image data, and can include performing a computer based tissue segmentation of the MRI rodent head image data, the segmentation outputting as a result, a three-dimensional (3D) tissue topology data representing boundary topologies of different tissue types of the rodent head tissues. The example method can include storing the 3D tissue topology data in the data memory, and computer-based generating, based on the 3D tissue topology data in the data memory and CT imaging rodent head data image data, 3D surface models of the different tissue types among the rodent head tissues. The example method can include, in the generating of the 3D surface models, a computer based labeling of the different tissue types of the rat head tissues, the labeling forming a 3D label map that represents different tissues appearing in the MRI rodent head image, and a computer-based generating, based on the 3D label map, 3D surface models of the different tissue types among the rodent head tissues, encoded as Surface Triangle Language (STL) or equivalent format files. The example method can include a computer-based refining of the STL or equivalent format files encoding the 3D surface models of the different types of tissue, and converting of a result of the refining to a simulation process compliant file format encoding of the 3D surface models.

According to one or more other embodiments, an example anatomically accurate rodent brain phantom can include a plurality of layers that, in combination, form a structure having a three-dimensional geometry that mimics a three-dimensional geometry of a rodent brain, wherein the configured to mimic respective brain structures including at least two of grey matter, white matter, and cerebrospinal fluid, wherein the layers comprise a conductive material comprising polydimethyl-siloxane (PDMS) and carbon nanotubes (CNTs).

According to one or more further embodiments, an anatomically accurate rodent brain phantom can include a plurality of layers configured to mimic respective brain structures including at least two of grey matter, white matter, and cerebrospinal fluid, wherein the brain structures are formed from a conductive material comprising polylactic acid (PLA).

In various embodiments, a method can provide a producing of an anatomically accurate rodent brain phantom, and the method can include forming an anatomically accurate inner shell and an outer shell that mimic an inner surface and an outer surface of a brain structure. The method according to the various embodiments can also include pouring a first conductive material comprising polydimethyl-siloxane (PDMS) and carbon nanotubes in between the inner shell and the outer shell, curing the first conductive material, and removing the inner shell and the outer shell to provide a brain phantom of said brain structure. The method according to the various embodiments can include forming one or more additional layers by pouring a second conductive material comprising polydimethyl-siloxane (PDMS) and carbon nanotubes, which can be between either at least one additional anatomically accurate shell and an existing layer of the brain phantom, or two existing layers of the brain phantom. The method according to various embodiments can also include curing the second conductive material, and responsive to an additional shell being used in the pouring step, removing the at least one additional shell.

In accordance to one or more further embodiments, a method can provide a producing of an anatomically accurate rodent brain phantom, and can include 3D printing a plurality of layers configured to mimic respective brain structures including at least two of grey matter, white matter, and cerebrospinal fluid, wherein the brain structures are formed from a conductive material comprising polylactic acid (PLA).

This Summary identifies example features and aspects and is not an exclusive or exhaustive description of disclosed subject matter. Whether features or aspects are included in or omitted from this Summary is not intended as indicative of relative importance of such features or aspects. Additional features are described, explicitly and implicitly, as will be understood by persons of skill in the pertinent arts upon reading the following detailed description and viewing the drawings, which form a part thereof.

### BRIEF DESCRIPTION OF THE DRAWINGS

FIG. 1 shows a logic flow diagram of an example process in constructing an anatomically accurate rat head model, in a brain phantom product process according to various embodiments of this disclosure.

FIGS. 2A-2C show exemplary Magnetic Resonance Images (MRIs) of an example rat brain, in an example tissue segmentation stage or phase in a brain phantom product process according to various embodiments, wherein FIG. 2A shows a transverse MRI slice of the example rat brain, FIG. 2B shows a sagittal MRI slice of the example rat brain, and FIG. 2C shows a coronal slice of the example rat brain.

FIGS. 3A-3D show exemplary views of an example anatomically accurate head model of an example rat head, in a brain phantom product process according to various embodiments, wherein FIG. 3A shows a meshed transverse view, FIG. 3B shows a meshed sagittal view, FIG. 3C shows a sagittal view, and FIG. 3D shows a coronal view of the example rat head, the coronal view showing skin, skull, grey matter, white matter and cerebrospinal fluid of the example.

FIGS. 4A-4D show structures and operations, illustrating an example progression in a brain phantom product process according to various embodiments, wherein FIG. 4A shows an example pair of complementary 3D-printed shells for assembly into an example two-piece 3D printed injection mold; FIG. 4B shows a stirring or mixing in a low viscosity phase of an example composite PDMS and CNT polymer; FIG. 4C shows an example injecting, in an example medium viscosity phase injecting the polymer into the mold; and FIG. 4D curing the polymer in a vacuum chamber.

FIGS. 5A-5E. Exemplary castings and 3D printed rodent brain phantoms in example practices of brain phantom product process according to some embodiments of the disclosure, wherein FIG. 5A shows a p[perspective view of an injection molded 3D rat brain phantom; FIG. 5B shows a perspective view of an inverted 3D printed skull with an open base aperture; FIG. 5C shows a perspective view of a partially separated two-piece 3D printed skull with the FIG. 5A phantom rat brain I the skull interior; FIG. 5D shows a perspective view of the FIG. 5C 3D printed skull, in an assembled, closed state; and FIG. 5E shows another perspective view of the 3D printed rat skull.

FIG. 6 illustrates a simplified logic schematic of a computing system on which example methods and processes according to various embodiments can be supported and practiced.

### DETAILED DESCRIPTION

Embodiments of the disclosure provide for the use of magnetic resonance imaging (MRI) and computer tomography (CT) images to create individualized tissue-segmented rodent brain models. Exemplary rodents include rats, mice, squirrels, hamsters, and guinea pigs. The systems and methods disclosed herein provide a flexible, semi-automated way to segment and handle MRI images for rodent models. The combined use of MRI and CT scan images may improve the model in areas where the MRI resolution or clarity is not sufficient. The disclosed models may then be used in the preparation of physical rodent brain phantoms which may be used, for example and without limitation, for testing Transcranial Magnetic Stimulation (TMS) or neuromodulation techniques.

TMS is a non-invasive procedure in which a TMS device, which includes an arrangement of wound coils connectable to a computer-controlled current source, is placed against the outside surface (e.g., skin) of the head, whereupon a time-varying electrical current is passed through the coils, producing one or more time-varying magnetic fields. The time-varying magnetic fields penetrate into the underlying brain tissue and act as a stimulus to a region of the brain. The treatment may be used to stimulate regions associated with mood disorders, for example. To reach the target tissue site, the stimulatory signal (e.g., the magnetic field) must traverse skin tissue, bone tissue (skull), cerebrospinal fluid, and some amount of neural tissue (brain). The stimulus may also reach the cerebellum to some extent. In order to improve the effectiveness of TMS, it is desirable if not essential to understand how the various operational parameters, e.g., feed current magnitude and oscillation frequency, and structural parameters, e.g., coil geometry and arrangements, of the TMS device affect different areas or parts of the patient's anatomy. Various exemplary embodiments can significantly assist in obtaining such understanding, at reasonable costs and without crossing into ethical concerns, understanding by providing methods for rapid turn-around, economical production a 3D anatomically realistic brain phantom (“phantom” for short).

According to various embodiments, a production process can comprise a head/brain modelling process, followed by a phantom fabrication process. Process implementations according to one or more embodiments can also include file conversions, e.g., a modelling file format-to-fabrication file format conversion process between the head/brain modelling process and the phantom fabrication process. According to various embodiments, the head/brain modelling process can include an imaging process, e.g., MRI imaging of a rodent head or a combination MRI and CT imaging of the head, followed by a tissue segmentation process. In one or more embodiments, the tissue segmentation process can be configured to segment the 3D MRI into a 3D grey matter (GM) segment, a 3D white matter (WM) segment, and a 3D cerebrospinal fluid (CSF) segment. According to various embodiments, the tissue segmentation process can be fully automated. In one or more embodiments, the tissue segmentation process can use a combination of automated segmentation processes and corrections or adjustments. Corrections can be based, for example, on a template.

In one or more embodiments, an end output of the tissue segmentation process can be, for example, in a conventional format such as, but not limited to, Neuroimaging Informatics Technology Initiative (NIFTI). According to various embodiments, the file format of the tissue segmentation process can be converted into one or more fabrication file formats such as, but not limited to STL.

In an example tissue segmentation process according to various embodiments, process logic blocks or sections can include a computer-based coregistering process section and what can be termed, for purposes of description, a computer-based actual segmentation process section. In an embodiment, these process sections can use a template, for example and without limitation, the SIGMA rat brain template. These and other process sections, which are described in more detail in later sections of this disclosure, can be implemented by a particularly configured digital processing resource, e.g., and without limitation, an appropriately configured, off-the-shelf programmable digital computer resource. Such configuration can include, for example and without limitation, particular computer-executable instructions, logically arranged, e.g., as a palette or “toolbox” of different executable instruction modules, that can provide the coregistering process section and the actual segmentation process section. This can include aligning the image to the template and creating, for this example, integer 3 separate gray matter, white matter, and cerebrospinal fluid NIFTI images.

In one or more embodiments, the fully automatic coregistering process and the fully automatic actual segmentation process can be configured to receive, for example and without limitation, various segmentation parameters, and for some application, these can enable or provide improved accuracy to either among or to both the fully automatic coregistering process and the fully automatic actual segmentation process. The various segmentation parameters can be provided by or implemented by, for example and without limitation, a local segmentation initialization resource. In one or more embodiments, implementations can include pre-stored segmentation parameters, e.g., stored in a segmentation parameter knowledge base local storage knowledge base, s an external source, various segmentation parameters. According to various embodiments, computer-based automatic processes (once the segmentation parameters are manually set) based on the SIGMA rat brain template. In the STL generation step, the software automatically creates a 3D model from the label map based on generation parameters (such as decimation or smoothing). Some aspects of the STL refinement process (such as automatic detection of holes, overlapping sections, and other errors) are done fully by the software. The finite element simulation is also automatic once the simulation parameters are set. This includes the calculations of the E and B fields as well as the visualizations of the simulations.

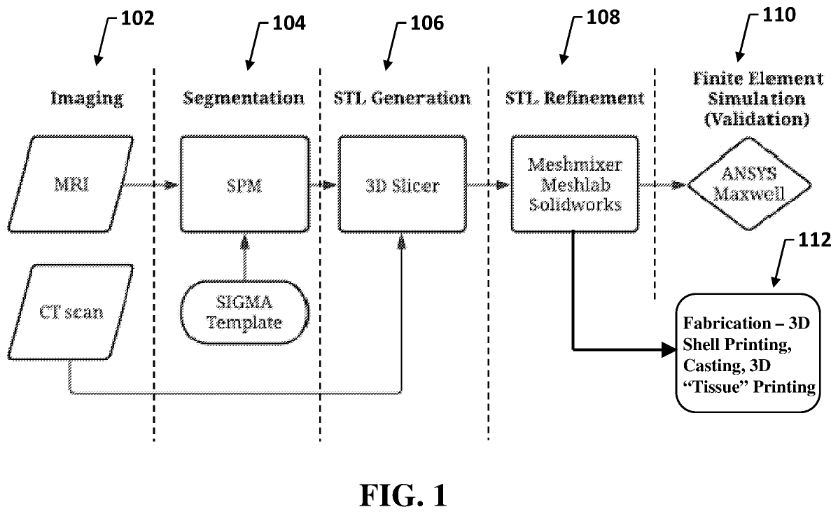

FIG. 1 shows a diagram of a logic flow 100 of example operations in a process, (hereinafter “process flow 100”), in constructing an anatomically accurate rat head model, in a brain phantom product process according to various embodiments of this disclosure. According to various embodiments, operations in the process flow 100 can include imaging 102, e.g., of the rodent head. The imaging 102 can include, for example, MRI, or a combination of MRI and CT.

In an embodiment, either immediately upon or sometime after the MRI imaging data from the imaging 102 of, for example, the subject rat head, is available to the tissue segmentation 104 section of the process flow, operations of the tissue segmentation 104 section can commence. In accordance with various embodiments, there may be various different types or ranges of time delay between performing the imaging 102 of the subject rat head and commencing the tissue segmentation 104. Determiners of such delay can include, but are not limited to, i) design choice; ii) operator choice; iii) whether resources for performing the MRI imaging component of the imaging 102 and resources for performing the CT imaging component of the imaging 102 are controlled by the same entity; iv) whether resources for performing the MRI imaging component of the imaging 102 and resources for performing the CT imaging component of the imaging 102 are geographically proximal one another, e.g., controlled by and co-located on a premise of a single entity.

As illustration, assume a first example, in which resources for performing the imaging 102, and resources for performing the tissue segmentation 104, as well as resources for performing other logic sections of the process flow 100, which are described in more detail in later paragraphs, are controlled by and co-located on a premise of a single entity. Further assume a specific example implementation according to such embodiments in which the respective controller logic for the MRI resource, and controller logic for the CT resource, as well as digital processing resources for the tissue segmentation 104, the STL generation 106, and STL refinement 108 can be connected to a common intra-entity network bus, for or instances of performing the process flow 100 in accordance with one or more embodiments,

According to various embodiments, operations in tissue segmentation 104 can include automated segmentation processes, such as but not limited to Statistical Parametric Mapping (SPM), which is readily available as a free and open-source MATLAB-based computer tissue segmentation tool, directed to brain image analysis. In accordance with various embodiments, and as shown in FIG. 1, component processes operations in tissue segmentation 104 can include alignment of the MRI of the brain and surrounding tissue the origin of the SIGMA rat brain atlas. Sub operations in tissue segmentation can include coregistering the file to the SIGMA atlas, which can involve the automatic orientation of the images to match the template. Coregistering and origin alignment can be omitted, but inclusion of these operations can provide statistically more accurate segmentation, as well as reduction of artifacts, and reduction of inaccurate solutions.

In paragraphs that follow, there is description of an example computer-based toolbox of exemplary computer-based tools, and combinations thereof that according to one or more embodiments can provide automatic processing implementation of process logic blocks and sections in the flow 100. Systems according to various disclosed embodiments can include, as described in more detail later in later paragraphs, digital processing resources. Functional features of the digital processing resources can include, but are not limited to, one or more hardware processors, e.g., microprocessors or microprocessor arrays, with or without supplemental logic resources, that can be coupled via a logic bus to a data memory resource and an instruction memory resource. To avoid description of details not relevant to concepts of this disclosure, and not likely to substantively assist persons of ordinary skill in the pertinent arts in obtaining an understanding of such concepts sufficient to practice its embodiments, example individual implementations of the various exemplary computer-based tools is omitted. In an embodiment, a general implementation that can be readily adapted by such persons for each of the computer-based tools can b, for each tool or for each of a subset of the tools, a respective a tool-specific block of, or collection of processor executable instructions, e.g., instruction “modules” that, when executed by the hardware processor, cause the processor to perform in accordance with the functionalities of tool. For various of the tools, description identifies particular off-the-shelf software products available from commercial vendors that can be used as implementations.

Operations in the tissue segmentation 104 can include, without limitation, segmenting the MRI into respective tissue classes. Implementations of the tissue segmenting 104 can but do not necessarily use, for example, SIGMA tissue probability maps (TPMs). Example implementations can also use SPM's Old Segment feature. Operations in the tissue segmentation 104 can include registering the MRI k to the original image for correct orientation. The tissue segmentation files can be cased, for example and without limitation, in NIFTI format.

In practices according to various embodiments, operations in tissue segmentation 104 can include coregistering the target image to the template coordinate space. Such embodiments' coregistering feature of the tissue segmentation 104 can provide, for example, improvement in alignment and the origin placement within the coordinate space. The embodiments' coregistering feature can also statistically reduce error from variation in brain structure as an arise, for example, from the template not exactly matching the target image due to natural variation.

Referring to FIG. 1, in accordance with various embodiments, operations in the process flow 100 can also include generation 106 of 3D files for image analysis phantom structure files. Example formats or protocols of such files that may be employed can include, but are not limited to, STL files. In various embodiments, computer-based tools for the generation 106 of phantom structure files can include and can utilize a tool such as, but not limited to, 3D Slicer. Functionalities of a tool such as 3D Slicer, in the generation 106, can include opening of NIFTI or equivalent files of GM, WM, CSF, and CT scan of the skull. Selection of brain tissue from each MRI image can use, for example, and without limitation, a label maker tool. According to various embodiments, features of the label maker tool can include, but are not limited to, automatic creating of a 3-dimensional label map that can represent various different tissues in an image. Features and functionalities of the label maker tool can also include a thresholding tool. In an embodiment, an implementation of the converting of the labels into 3D STL files can use, for example and without limitation, model maker. According to various embodiments, functionalities of the model maker tools can include, but are not limited to, automatic creating of 3-dimensional surface models from label maps, e.g., the segmented image data from the label maker. The above-identified thresholding tool in the label maker was used to separate the skull and surrounding tissue. In an embodiment, discontinuous islands can be removed from the label map, using e.g., and without limitation, the Island tool, Benefits of such removal can include, for example, provision of a continuous label of the tissues. According to various embodiments, general functionalities of the island tool can include, but is not limit to, creating of a unique label for each connected/continuous region in a given label map. Examples of specific component tools or functionalities of the island tool can include, without limitation, a tool that splits the STL or other format file into regions that can be referred to as “identify islands,” and can include a “remove islands” tool which identify different continuous sections and remove all but the largest continuous section, respectively

According to various embodiments, decimation can be used. Persons of ordinary skill in the pertinent arts, upon reading this disclosure in its entirety, can readily determine whether or not to use decimation and, if used, can determine appropriate decimation rates or ranges of such rates, without undue experimentation. For purposes of illustration, and not to be understood as any limitation or preference, one example decimation rate can be 0.5. The inventors, as of the relevant filing date, are without knowledge any embodiment-specific guidelines for setting the decimation rate, e.g., particular embodiment-specific criteria, or considerations, or weightings thereof. The inventors believe that persons of ordinary skill in the pertinent arts, upon reading this disclosure in its entirety and applying relevant engineering know-how and knowledge of conventional techniques of selecting such decimation rates that are possessed by such persons, can readily identify appropriate decimation without undue experimentation.

According to various embodiments, smoothing iterations can also be used, e.g., to reduce file size. For purposes of illustration, and not to be understood as any limitation or preference, one example smoothing can include 20-40 Sinc smoothing iterations. The inventors, as of the relevant filing date, are without knowledge any embodiment-specific guidelines for setting the smoothing parameters, e.g., particular embodiment-specific criteria, or considerations, or weightings thereof. The inventors believe that persons of ordinary skill in the pertinent arts, upon reading this disclosure in its entirety and applying relevant engineering know-how and knowledge of conventional techniques of setting smoothing parameters that are possessed by such persons, can readily identify appropriate smoothing parameters without undue experimentation.

Referring still to FIG. 1, in accordance with various embodiments, operations in the process flow 100 can include STL refinement 108. Example operations in STL refinement 108 can include, but are not limited to, reduction of overall STL file size and fixing or correction of larger model errors. Such STL refinement 108 operations can be performed, for example, via Meshmixer or an equivalent tool. Alternatives to Meshmixer can include, without limitation, Meshlab. Another example is Solidworks. Operations in STL refinement 108 can further include, for example, removal of self-intersecting faces, fixing non-manifold edges, and other minor STL repairs. Such operations can be performed, for example, via Meshlab or an equivalent. The above-described operations, e.g., removal of self-intersecting faces, fixing non-manifold edges, and other minor STL repairs can verify validity of the model used in subsequent finite element simulation 110 operations. According to one or more embodiments, such STL refinement 108 operations can include import diagnostics and exporting in STEP format. The resulting files can be final files for the GM, WM, CSF, skull, and tissue. Implementation can include, for example and without limitation, SolidWorks.

FIGS. 3A-3D shows exemplary views of an example anatomically accurate head model of an illustrative rat head, such as may be generated by the STL refinement 108 for use in finite element simulation 110 operations in a brain phantom product process according to various embodiments of this disclosure. FIG. 3A shows a meshed transverse view of the example anatomically accurate head model of rat, FIG. 3B shows a meshed sagittal view of the example anatomically accurate head model, FIG. 3C shows a sagittal view, and FIG. 3D shoes a coronal view of the rat's brain, showing skin, skull, grey matter, white matter, and cerebrospinal fluid.

According to various embodiments, operations in finite element simulation 110 can include combining the anatomically accurate individualized brain model output from the STL refinement 108 with a model of a TMS coil. For brevity, the phrase “model of the TMS coil” will hereinafter be alternatively recited as “TMS coil model.”. In an embodiment, TMS coil(s) emulated by the TMS coil model can be, for example and without limitation, a TMS coil designed for small animals, such as represented by the FIG. 1 example ANSYS Maxwell 3D modeler.

In one or more embodiments, practices of the finite element simulation 110 can include modeling an enclosure surrounding the coil and brain, which can serve as air in the simulation). Individual electromagnetic properties of each tissue, for use in the finite element simulation 110 can be assigned based, for example, on previous literature. An illustrative example of values obtained from previous literature is shown in Table 1. Regarding configurations of the TMS coil, and models of same, examples can include, but are not limited to, a copper wire overlapping “FIG. 8” coil. Such an example, or another example configuration of one or more TMS coils, can be modeled and the model imported for combination with the above-described model of the rat brain.

Finite element simulation 110 can then be run, and in an example can include a modeled feed current to the modeled TMS coil or combination of TMS coils. As will be understood by persons of ordinary skill upon reading this disclosure, specific feed current amplitudes and oscillation frequencies for finite element simulation 110 can be application-specific, and selection factors can include, for example and without limitation, geometry and arrangement of the TMS coils, dimensions of the modeled brain and encasement skull of same. For purposes of illustration, in the context of a modeled rat brain output from STL refinement 108 or directly from the generation 106, an example feed current amplitude can be, without limitation, 5 kA, and an example feed current oscillation frequency can be, example feed current amplitude can be, without limitation, 2.5 kHz. A simulation tool such as, but not limited to, a Maxwell 3D module using ANSYS finite element simulation software can then simulate the magnetic flux density and induced electric field in the rat brain models.

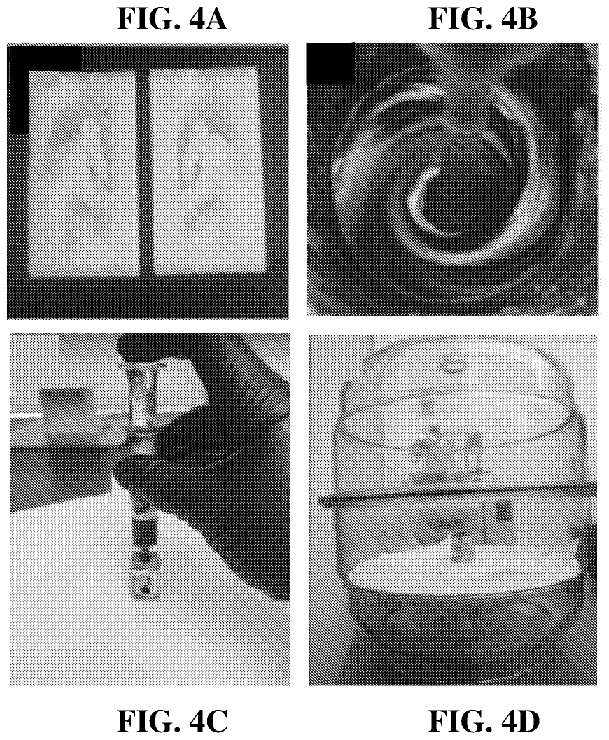

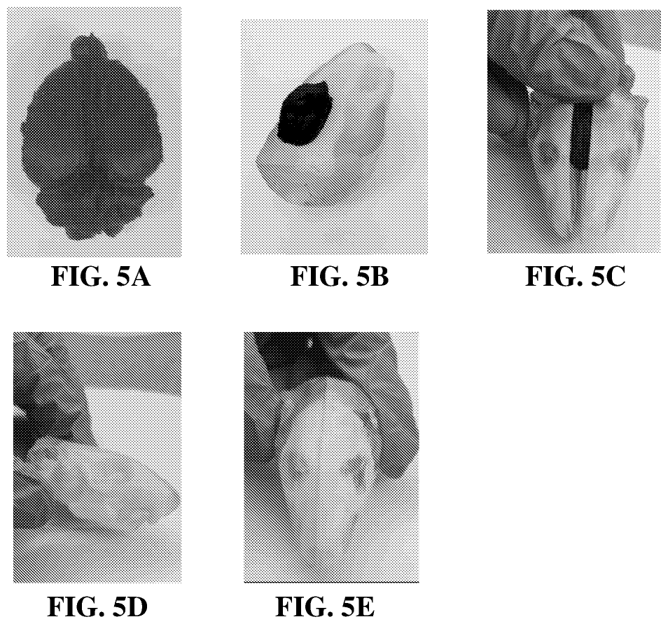

According to various embodiments, operations in the process flow 100 can include fabrication 112 of a brain phantom, using the modeled rat brain output from STL refinement 108. Features and example operations and implementations in the fabrication will be described in more detail in subsequent paragraphs, and will include reference to FIGS. 4A-4D and 5A-5C.

Referring to FIGS. 4A-4D, for some exemplary phantoms according to various disclosed embodiments, shells can be 3D printed for each tissue layer of the brain. After 3D printing, spaced between pairs of the shells can be filled with a conductive material (e.g., silicon or PDMS with nanoparticles) that is configured to mimic the conductive properties of brain tissue. Different conductive material compositions may be used for different tissues (e.g., white matter vs. grey matter vs. CNF; brain matter vs. bone vs. skin). In accordance with various embodiments, one or more “brain tissue” layers can be 3D printed, using for example the above-described conductive material. In one or more of such embodiments, the 3D printing can use a prior cast “brain tissue” as a support or substrate for the 3D printing of additional “brain tissue.” In other embodiments, a 3D printed shell can be used as a support or substrate for the 3D printing of additional “brain tissue.”

FIGS. 5A-5C show exemplary castings and 3D printed rodent brain phantoms in example practices of brain phantom product process according to some embodiments of the disclosure. FIG. 5A shows a 3D-printed shell which defines the boundary geometry of a region of rat grey matter. FIG. 5B shows a 3D-printed shell which defines the boundary geometry of a region of rat white matter. Suitable materials for shells are those which are 3D printable, shape resilient, and cable of being dissolved by subsequent chemical treatment. An exemplary material meeting these criteria may be a thermoplastic polymer such as Acrylonitrile butadiene styrene (ABS). The shell may be used as mold for casting the conductive material which belongs in the phantom at the end of the manufacturing process. FIG. 5C shows cast material of rat grey matter after it has been cured and the shell removed.

Generally, casting of a brain phantom may be facilitated by making pairs of shells as casting molds for respective parts of the brain, each pair having a top part (i.e., upper part) and a bottom part (i.e., lower part). An exemplary process is disclosed in U.S. Patent Publication 2019/0057623 incorporated herein by reference. This approach may be used for some elements of the brain phantom and not for others. For instance, using a pair of shells (one upper part and one lower part) may be especially well suited for casting grey matter, cerebrospinal fluid (CSF), bone, and skin. For parts such as the ventricles and cerebellum, a single shell may be used. In some instances, a shell (be it upper or lower) may technically consist of two shells, respectively referred to as an inner shell and outer shell. As will be discussed below, for example, grey matter may be cast using inner and outer upper shells as well as inner and outer lower shells.

In one or more embodiments, polyjet 3D printing can be used to form the skin and bone layers. In various embodiments, shells can be printed using, for example, SR30 support material and PLA, as opposed to the human brain phantom which can use, as described above, PVA. In another example, according to one or more embodiments, rat phantom fabrication can employ curing one layer on top of the last using one dissolvable set of shells for the inner mass and a reusable set of shells for the outer.

3D printing is an exemplary means for producing the shells. Advantages of 3D printing the shells, as opposed to machining the molds from aluminum or other metal stock, include cost effectiveness, e.g., lower fabrication time, and lower complexity fabrication equipment. An exemplary material for the 3D printing process is an ABS material. 3D printing may require the printing of supporting structures which do not actually have any anatomical analog. In such case these supporting structures may be removed by, for example, chemically dissolving the parts (e.g., with acetone for ABS). Next the conductive “tissue” material is poured between shells (e.g., between and upper and lower shell pair, and/or between an inner and outer shell pair), and permitted to cure. The curing process may involve time during which the chemical composition of the “tissue” material reacts and sets. The curing process may involve exposing the “tissue” material to various forms of electromagnetic radiation, e.g., UV radiation for curing of radical-type (acrylates) and cationic type (epoxides, vinyl ethers) monomers. In one or more embodiments, the curing can be performed by sitting for a finite duration in a room temperature. Also, in one or more embodiments using conductive fibers, e.g., graphene, in the polymer composite, a magnetic field can be used to align such fibers.

After the conductive “tissue” material cures, the shells and the conductive material are placed in an appropriate chemical bath (e.g., acetone) to dissolve all remaining shell material (e.g., ABS), leaving only the cast “tissue” material for the phantom behind. Alternative shell removal techniques may also be used in embodiments. For instance, shells may in some cases be physically fractured or broken and the resulting fragments removed (without any dissolving necessary). The mold-making and casting are repeated for subsequent parts.

As will be discussed in greater detail below, for some tissue structures a prior casting may be used in place of one or more shells. For example, an additional tissue structure, e.g., layer, can be 3D printed on a prior casting of a structurally “inner” tissue. As a result some tissue structures of the phantom can be produced using two or more shells, some with only one shell, and some without using any shells. Advantages of this approach are many. Fewer shells means less 3D printing which means lower costs of production. Using a prior casting of an existing part as a “mold” surface for the next part also means the two tissues will intimately share a surface boundary with precise conformance, which can avoid or reduce the probability of gaps between phantom layers. This is beneficial, as such gaps can negatively affect the conductive behavior across the material-to-material boundary.

At the conclusion of the process, all shells have been removed and a multi-layered brain phantom remains and is ready for use.

It will be appreciated by ones of skill in the art that reference to “a shell” in the singular may be understood as indicative of multiple shells, for example a pair or two pairs of shells. Similarly, single shells may be used in some instances where a plurality is described. The details on shell pairing are already described above.

The end result of the process is a complete brain phantom containing one or more “tissue” structures, for example 2, 3, 4, 5, 6, or more differentiated “tissues”. The tissues may include grey matter, white matter, cerebrospinal fluid (CSF, including that which surrounds the brain and that which is contained in the ventricles), cerebellum, bone, and skin. Note that CSF may be referred to as a tissue or structure herein despite technically being a fluid in living organisms. Note also the ventricles may be referred to as a tissue or structure despite technically being cavities in living organisms. In the context of brain phantoms, both CSF and ventricles (which in living organisms are filled with CSF) may be simulated with solid or semisolid materials.

In the above descriptions for manufacturing brain phantoms, the materials used for the phantom layers are generally described as conductive materials. Addressing the materials directly, an exemplary conductive material is a silicon or silicone based compound (a compound containing silicon, Si) or PDMS with one or more of graphite, multi walled or single walled carbon nanotubes (MWCNT/SWCNT) that is capable of mimicking the electrical conductive properties of different brain tissues based on the respective amounts of these constituents. The conductivity of layers of an exemplary phantom may be in the range between 0.2-3.0 Siemens-per-meter (Sm⁻¹). For the skin layer the conductivity range may be lower, e.g., and without limitation, as low as 0.1 Sm⁻¹. In some other embodiments the layers may each be in the range of, for example and without limitation, 0.2-1.8 Sm⁻¹. In a particular example, the electrical conductivity of different brain tissue that was matched in a phantom was as follows: ventricles & CSF=1.77 Sm⁻¹, GM=0.23 Sm⁻¹, WM=0.24 Sm⁻¹, and cerebellum=0.65 Sm⁻¹. It will be understood that these specific values of conductivity are for only purposes of illustration, and are not intended as limitations or as preferences in practices in accordance with disclosed embodiments.

An “anatomically accurate” brain phantom mimics pertinent characteristics of the brain of a living organism, e.g., a mammalian brain (e.g., a rodent brain). The scope of “pertinent characteristic,” i.e., the domain or list of metrics of “anatomically accurate,” can be application-specific. Examples can include, but are not limited to, three dimensional geometry (e.g., sizes, relative sizes, dimensions, relative dimensions, locations or positions, relative locations or positions, etc.) of the phantom matches or substantially matches the three dimensional geometry of a real brain (e.g., an actual rodent brain). The domain of anatomically accurate may include one or more electrical properties (e.g., electrical conductivity) of the brain phantom match or substantially match one or more electrical properties of a real brain (e.g., an actual rodent brain).

Anatomically accurate may mean one or more material properties (e.g., mass density, viscosity, etc.) of the brain phantom match or substantially match one or more material properties of a real brain (e.g., an actual rodent brain). A brain phantom may match or substantially match a real brain if at least one layer/structure of the brain phantom matches or substantially matches the corresponding real brain structure. A brain phantom may match or substantially match a real brain only if all the layers/structures of the brain phantom match or substantially match the real brain. Table 1 below presents exemplary but non-limiting material properties which may be used in a computer simulation or physical brain phantom which is anatomically accurate.

TABLE 1
Material properties for simulation or physical brain phantoms

| | Mass Density | Electrical | Relative |
|---|---|---|---|
| Structure | (kg/m³) | Conductivity (S/m) | Permittivity |
| Skin | 1109 | 0.17 | 1 |
| Skull | 1908 | 0.32 | 1 |
| CSF/Ventricles | 1007 | 1.7765 | 1 |
| Grey Matter | 1044.5 | 0.239149 | 1 |
| White Matter | 1041 | 0.26507 | 1 |
| Cerebellum | 1045 | 0.659667 | 1 |

An anatomically accurate brain phantom may be produced with the conductivities of the grey matter and white matter in the range of 0.1 to 0.5 Sm⁻¹. Different conductivities may be used for different structures/layers/regions of the brain phantom. To achieve different conductivities, different composite polymers may be prepared and used. For example, exemplary brain phantoms or structures/layers thereof may comprise a composite polymer of a silicon-based compound (e.g., PDMS) and carbon nanotubes (in particular multi-walled carbon nanotubes, MWCNTs) with the conductivity/resistivity varied among the structures/layers by variable weight percent (wt %) of the MWCNTs. Table 2 presents the relationship between resistivity and composition of MWCNTs in PDMS.

TABLE 2
Relationship between resistivity and composition of MWCNTs

| wt % of CNT in PDMS | Resistivity (ohm/cm) |
|---|---|
| 10.5 | 1000 |
| 11.5 | 500 |
| 12.5 | 300 |
| 15.3 | 35 |

Resistivity of 300-400 ohms/cm corresponds to 0.3-0.5 S/m (an exemplary target value range for WM and GM). In some embodiments, the amount of CNTs in the composition varies from about 6-12 wt %, e.g. about 8-10 wt %. For example, the grey matter may have 8-10 wt % CNTs, and the white matter may have 9-12 wt % CNTs. CSF is a fluid that will be filled between grey matter and the skull. The fluid composition can be from deionized water and salt that matches the conductivity of 1.5-2 S/m.

In additional embodiments, the brain phantom as described herein is composed of 3D-printed polylactic acid (PLA) filaments. The conductance of the 3D-printed PLA may vary with the infill percentage which refers to the density of the printed pattern. A phantom formed from PLA may be more precise in the conductivity values of the different layers and will not change with respect to the curing time or applied forces. While conductive PLA may be more stable with respect to the conductivity in time, it may not represent the mechanical properties of the brain that closely. Thus, the PLA phantom may be more useful to perform measurements of the induced electric field in the brain surface, where the mechanical properties are not as relevant since measuring probes do not need to penetrate the surface. In some embodiments, the respective infill percentages of the plurality of layers can be brain structure specific, i.e., set according to the brain structure of which the layer will be a constituent portion. For example, layers that are portions of phantom regions mimicking grey matter may have an infill percentage of 80-90%, and layers mimicking white matter may have an infill percentage of 80-95%.

The electrical conductivities of the different layers or structures of exemplary brain phantoms can be varied with respect to one another by varying one or more of the materials or compositional ratios or infill percentages with respect to the other layers/structures. For example, different layers or structures may be configured to have different electrical conductivities based on nanotubes of different lengths in one layer versus another layer (e.g., shorter in one layer versus longer in another layer). Different layers or structures may be configured to have different electrical conductivities based on different materials for the nanotubes in one layer versus another layer (e.g., carbon versus silver). Different layers or structures may be configured to have different electrical conductivities based on different types of nanotubes in one layer versus another layer (e.g., single walled versus multi walled nanotubes).

The phantoms described herein are suitable for use in simulations, tests, and experimentation, for example, relating to open surgery on the brain (e.g., where the skin and bone are removed). The phantoms may also be used for simulated and experimental verification of induced electric fields in rodent brains, for the creation of new coils for TMS and other neuromodulation techniques, or for rodent studies of neurological conditions such as Parkinson's disease. Multiple procedures (e.g., a series of procedures) may be performed on the same brain phantom. Neuromodulation procedures may include one or more of TMS, transcranial direct current stimulation (tDCS), or deep brain stimulation (DBS). Neuroimaging procedures may include MRI, for example. Other procedures, e.g., and without limitation CT, may also or alternatively be performed.

Where computer software is discussed herein, it should be understood that such software may be embodied in computer readable instructions which may be provided to one or more processors of a general purpose computer, special purpose computer, or other programmable data processing apparatus to produce a machine, such that the instructions, which execute via the processor of the computer or other programmable data processing apparatus, create means for implementing the functions/acts specified in the description above, in one of the flowcharts, and/or in one or more block diagram blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowcharts and/or block diagrams. Embodiments herein may comprise one or more computers, one or more processors, one or more computer readable storage media, and/or appropriate input/output devices therefore, as well as additional supporting hardware as necessary.

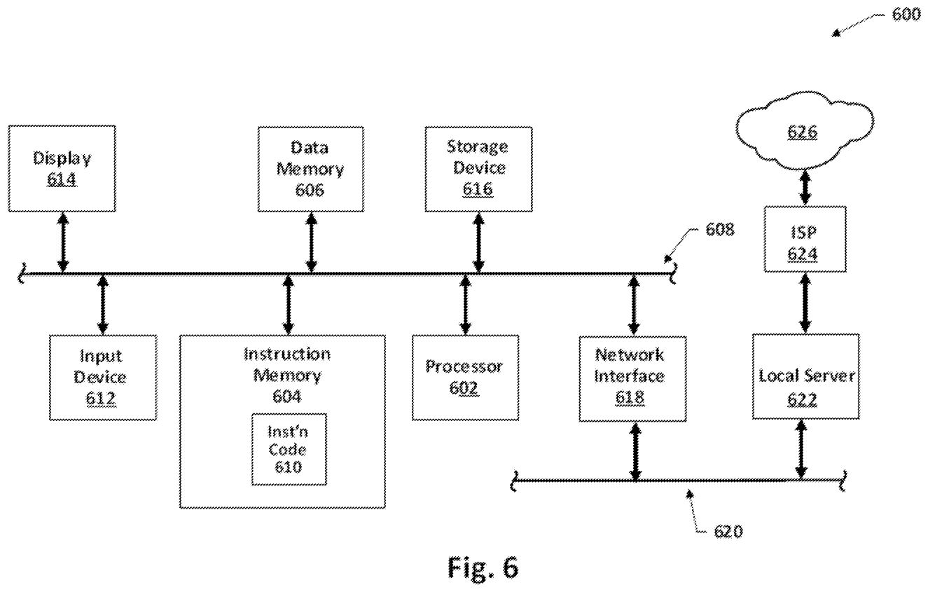

FIG. 6 illustrates a simplified logic schematic of a computing system 600 on which example methods and processes according to various embodiments can be supported, implemented, and/or practiced. The computer system 600 can include a hardware processor 602 communicatively coupled to an instruction memory 604 and to a data memory 606 by a bus 608. The instruction memory 604 can be configured to store, on at least a non-transitory computer readable medium, executable program code 610. The executable code 610 can implement processor 602 executable instructions and in various embodiments, at least portions of the executable instructions can be logically arranged as instruction modules. The computer system 600 can also include an input device 612, e.g. a mouse or a touchscreen aspect of a display screen, such as the example display 614. The computer system 600 can further include, connected to the bus 608, a mass storage 616, and a network interface 618. The network interface 618 can connect to a local, e.g., intra-office network 620. In an embodiment, the computer system 600 can also include, connected to the local network 620, a local server 622m which, e.g., via an ISP 624m, can access the Internet.

Non-transitory computer-readable media may be understood as a storage for the executable program code. Whereas a transitory computer-readable medium holds executable program code on the move, a non-transitory computer-readable medium is meant to hold executable program code at rest. Non-transitory computer-readable media may hold the software in its entirety, and for longer duration, compared to transitory computer-readable media that holds only a portion of the software and for a relatively short time. The term, “non-transitory computer-readable medium,” specifically excludes communication signals such as radio frequency signals in transit.

The following forms of storage exemplify non-transitory computer-readable media: removable storage such as a universal serial bus (USB) disk, a USB stick, a flash disk, a flash drive, a thumb drive, an external solid-state storage device (SSD), a compact flash card, a secure digital (SD) card, a diskette, a tape, a compact disc, an optical disc; secondary storage such as an internal hard drive, an internal SSD, internal flash memory, internal non-volatile memory, internal dynamic random-access memory (DRAM), read-only memory (ROM), random-access memory (RAM), and the like; and the primary storage of a computer system. Executable program code may therefore be understood to be a set of machine codes selected from the predefined native instruction set of codes. A given set of machine codes may be understood, generally, to constitute a module. A set of one or more modules may be understood to constitute an application program or “app.” An app may interact with the hardware processor directly or indirectly via an operating system. An app may be part of an operating system.

Unless the context indicates otherwise, block diagrams and flowcharts are exemplary and may involve fewer or greater number of blocks and/or a different order of items or steps. In some embodiments elements or steps may be concurrent, combined, or otherwise organized differently than is depicted or described.

Before exemplary embodiments of the present invention are described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely.” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### EXAMPLES

### Example 1. Physical Brain Phantom

FIG. 4A shows two molds formed by 3D printing, via 3D mold printing in the FIG. 1 process flow 100, based on STL files generated based on tissue segmentation results. The example molds were used to create two inverse cavities in SolidWorks®, that would later form the 3D printed molds for the rat brain phantom. The first, i.e., leftmost mold in FIG. 4A is an inverse of the surface topology of the rat grey matter tissue segment obtained by the tissue segmentation 104, and the second, i.e., rightmost mold, is an inverse of the full brain. Polydimethylsiloxane (PDMS) and Carbon nanotubes (CNT) were used to create a composite polymer that can have varying conductivity values depending on the weight percent of the CNT. CNT nanoparticles with the proper weight percent suspended within the PDMS form the composite polymer as a conductive grid that provides the composite with its conductive characteristic. To achieve the proper conductivity in the phantom, an example range of weight percent of CNT can be, but is not limited to, between 8% and 10%. In an embodiment, a process of mixing the PDMS and CNT composite can be described as having 3 distinct phase. The first may be referred to as a “low viscosity” phase, the second as a “medium viscosity” phase, and the third as a “high viscosity.” In medium viscosity phase the composite starts to thicken, and in the high viscosity phase when the stirrer doesn't create flows in the complex. Operations in the low viscosity phase can comprise mixing, e.g., stirring, a low viscosity mixture of CNT and PDMA, using proper ratios. A snapshot view of a low viscosity mixing is shown in FIG. 4B. After a duration of the low viscosity mixing, the mixture starts to thicken, and the mixing progresses to the medium viscosity phase. AN example operation in the a low viscosity mixing is shown in FIG. 4B. Operations in the low viscosity phase can comprise mixing, e.g., stirring, a low viscosity mixture of CNT and PDMS, using proper ratios. A non-limiting example operation in the medium viscosity phase, as shown in FIG. 4C, can comprise introducing, e.g., injecting the conductive polymer into the mold. A non-limiting example operation in the third phase, i.e., “high viscosity” comprises curing the polymer, and an example is shown in FIG. 4D. According to one or more embodiments, the curing can be in a vacuum chamber. The full mixing time can be, for example, roughly 10 minutes before adding the curing agent to the mixture.

Once the curing agent was mixed with the polymer, it was injected into the 3D printed grey matter mold (casting) and left to cure in a vacuum chamber. As the casting cures in the vacuum chamber, the air trapped in the polymer begins to escape and subsequently forces small amounts of the PDMS/CNT mixture to fill the gaps left. To ensure a solid inner volume without discontinuities, it is required to continuously inject more polymer into the mold for the first few minutes, or until it is no longer being extruded out. Once the grey matter volume was created, it was inserted into the second mold, and all steps after mold making were repeated. This method allows for the preparation of a brain phantom with multiple layers that fit together perfectly, each with their own typical conductivity values.

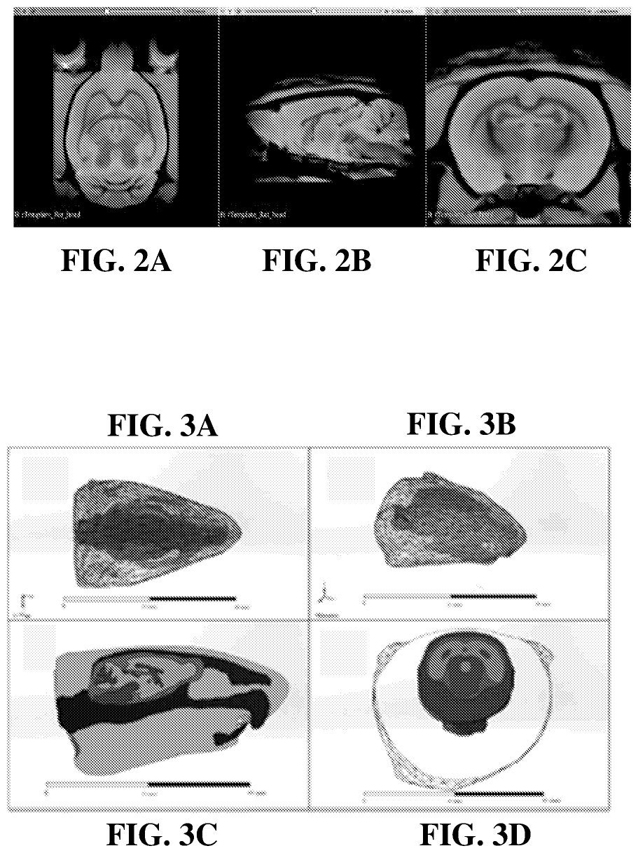

FIGS. 2A-2C demonstrate the results of mimicking the complex geometry of the rat brain using a molding process.

While exemplary embodiments have been disclosed herein, one skilled in the art will recognize that various changes and modifications may be made without departing from the scope of the invention as defined by the following claims.

We claim:

1. A method for constructing an anatomically correct model of a rodent head that is useable for testing or evaluating a transcranial magnetic stimulation (TMS) or other neuromodulation system or method, and useable for fabricating a brain phantom that mimics the rodent head, the method comprising:
receiving, and storing in a data memory of a programmable computer resource, a tissue imaging data for the rodent head, the tissue imaging data including a magnetic resonance imaging (MRI) rodent head image data and a computer tomography (CT) imaging rodent head data image data;
performing a computer based tissue segmentation of the MRI rodent head image data, the segmentation outputting as a result, a three-dimensional (3D) tissue topology data representing boundary topologies of different tissue types of the rodent head tissues;
storing the 3D tissue topology data in the data memory;
computer-based generating, based on the 3D tissue topology data in the data memory and CT imaging rodent head data image data, 3D surface models of the different tissue types among the rodent head tissues, by operations including
a computer based labeling of the different tissue types of the rat head tissues, the labeling forming a 3D label map that represents different tissues appearing in the MRI rodent head image, and
a computer-based generating, based on the 3D label map, 3D surface models of the different tissue types among the rodent head tissues, encoded as Surface Triangle Language (STL) or equivalent format files; and
computer-based refining of the STL or equivalent format files encoding the 3D surface models of the different types of tissue, and converting of a result of the refining to a simulation process compliant file format encoding of the 3D surface models.

2. The method of claim 1, wherein the computer based tissue segmentation of the MRI rodent head image data comprises a hardware processor of the programmable computer resource, coupled to the data memory and to an instruction memory by a bus, executing hardware processor executable segmentation instructions stored in the instruction memory that, when executed, cause the hardware processor to retrieve the MRI rodent head image data from the data memory, perform the tissue segmentation and generate the 3D tissue topology data

3. The method of claim 1, wherein the computer-based labeling of the different tissue types of the rat head tissues, the comprises the hardware processor of the programmable computer resource executing hardware processor executable label maker instructions stored in the instruction memory that, when executed, cause the hardware processor to perform:
retrieving the 3D tissue topology data from the data memory,
retrieving the CT imaging rodent head data from the data memory,
separating, based on a threshold, the skull and surrounding tissue, from the 3D tissue topology data and from the CT imaging rodent head data 3D rodent brain tissue data,
selecting, for, a result of the separating, brain tissue from 3D tissue topology data and from the CT imaging rodent head data CT imaging rodent brain tissue data, and
generating the label map based on a result of the selecting.

4. A method for finite element simulation of a TMS system or other neuromodulation system operating on a subject rodent head, comprising:
magnetic resonance imaging (MRI) of the subject rodent head image data and computer tomography (CT) imaging of the subject rodent head data, generating, MRI subject rodent head image data and CT imaging subject rodent head data;
generating, according to the method of claim 1, using the MRI subject rodent head image data as the MRI rodent head image data and the CT imaging subject rodent head data as the imaging rodent head data image data, the claim 1 simulation process compliant file format encoding of the 3D surface models;
storing in a data memory of a programmable processor of a finite element simulation resource.

5. An anatomically accurate rodent brain phantom, comprising a plurality of layers that, in combination, form a structure having a three-dimensional geometry that mimics a three-dimensional geometry of a rodent brain, wherein the configured to mimic respective brain structures including at least two of grey matter, white matter, and cerebrospinal fluid, wherein the layers comprise a conductive material comprising polydimethyl-siloxane (PDMS) and carbon nanotubes (CNTs).

6. The anatomically accurate rodent brain phantom of claim 5, wherein at least two layers among the plurality of layers have different wt % of CNTs with respect to one another and, based at least in part on the different wt % of CNTs with respect to one another, the at least two layers have mutually different electric conductivities.

7. The anatomically accurate rodent brain phantom of claim 6, wherein the plurality of layers contain 8-10 wt % CNTs.

8. The anatomically accurate rodent brain phantom of claim 5, wherein the plurality of layers are configured to have an electrical conductivity of 0.2 to 3.0 S/m to mimic an electrical conductivity of a brain structure.

9. An anatomically accurate rodent brain phantom, comprising a plurality of layers configured to mimic respective brain structures including at least two of grey matter, white matter, and cerebrospinal fluid, wherein the brain structures are formed from a conductive material comprising polylactic acid (PLA).

10. The anatomically accurate rodent brain phantom of claim 9, wherein at least some of the plurality of layers are configured to have different electric conductivities by having different infill percentages with respect to one another.

11. The anatomically accurate rodent brain phantom of claim 10, wherein the infill percentage of the plurality of layers ranges from 80-95%.

12. The anatomically accurate rodent brain phantom of claim 9, wherein the plurality of layers are configured to have an electrical conductivity of 0.2 to 3.0 S/m to mimic an electrical conductivity of a brain structure.

13. A method of producing an anatomically accurate rodent brain phantom, comprising
forming an anatomically accurate inner shell and an outer shell that mimic an inner surface and an outer surface of a brain structure;
pouring a first conductive material comprising polydimethyl-siloxane (PDMS) and carbon nanotubes in between the inner shell and the outer shell;
curing the first conductive material;
removing the inner shell and the outer shell to provide a brain phantom of said brain structure;
forming one or more additional layers by pouring a second conductive material comprising polydimethyl-siloxane (PDMS) and carbon nanotubes between either
at least one additional anatomically accurate shell and an existing layer of the brain phantom, or
two existing layers of the brain phantom;
curing the second conductive material; and
responsive to an additional shell being used in the pouring step, removing the at least one additional shell.

14. The method of claim 13, wherein the anatomically accurate rodent brain phantom is configured to mimic respective brain structures including at least two of grey matter, white matter, and cerebrospinal fluid.

15. The method of claim 13, further comprising configuring the one or more additional layers to have different conductivities with respect to one another by varying the wt % of CNTs from one layer to the next.

16. The method of claim 13, wherein the forming step comprises 3D printing the anatomically accurate inner and outer shells.

17. A method of producing an anatomically accurate rodent brain phantom, comprising 3D printing a plurality of layers configured to mimic respective brain structures including at least two of grey matter, white matter, and cerebrospinal fluid, wherein the brain structures are formed from a conductive material comprising polylactic acid (PLA).

18. The method of claim 17, further comprising configuring the plurality of layers to have different conductivities with respect to one another by varying the infill percentage from one layer to the next.
